(19) United States
(12) Patent Application Publication
(10) Pub. No.: US 2020/0317593 A1
(43) Pub. Date: Oct. 8, 2020

(54) LIGNIN CONVERSION TO PHENOLIC MOLECULES USING TRANSITION METAL CATALYSTS

(71) Applicant: SPERO RENEWABLES, LLC, Goleta, CA (US)

(72) Inventors: Ian M. KLEIN, Goleta, CA (US); Daniel H. COLLER, Goleta, CA (US)

(21) Appl. No.: 16/652,917

(22) PCT Filed: Nov. 30, 2018
(86) PCT No.: PCT/US2018/063351

§ 371 Date: Apr. 1, 2020

Related U.S. Application Data
(60) Provisional application No. 62/593,366, filed on Dec. 1, 2017.

(51) Int. Cl. C07C 41/44; B01J 35/04; B01J 23/755; B01J 21/18; B01J 8/02; B01J 8/04; C08G 59/06; C08L 63/00; B01D 11/02

(52) U.S. Cl. CPC C07C 41/44; B01J 35/04; B01J 23/755; B01J 21/18; B01J 8/0278; B01J 2208/021; C08G 59/063; C08L 63/00; B01D 11/0288; B01D 11/0219; C08L 2205/16; B01J 8/0492

(57) ABSTRACT

A process for processing lignin includes contacting a mixture comprising lignin and/or lignin-like molecules with a catalyst to form a reaction mixture, and producing one or more reaction products. The reaction mixture comprises one or more aliphatic alcohols, and the one or more reaction products are selected from the group consisting of: 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol), and mixtures thereof.

### CROSS-REFERENCE TO RELATED APPLICATIONS

The present application claims priority to U.S. Provisional Application No. 62/593,366 filed on Dec. 1, 2017 and entitled “Lignin Conversion to Phenolic Molecules Using Transition Metal Catalysts,” the disclosure of which is incorporated herein in its entirety.

### BACKGROUND

Lignin is the second most abundant material in biomass, comprising 15-30 wt %. Lignin is comprised of a network of monolignols such as coniferyl, sinapyl, and paracoumaryl alcohol. Although lignin is a potential source for aromatic compounds and polymers, the recalcitrant nature of lignin makes the selective and cost-effective production of high value and commodity chemicals difficult and problematic. Lignin is primarily used as a low value heat source as a result. As a result, many aromatic chemicals important to chemical, food and flavor, pharmaceutical, and polymer companies are continually sourced from non-renewable sources such as petrochemicals.

### SUMMARY

In some embodiments, a process includes contacting a mixture comprising lignin and/or lignin-like molecules with a catalyst to form a reaction mixture, and producing one or more reaction products. The reaction mixture comprises one or more aliphatic alcohols, and the one or more reaction products are selected from the group consisting of: 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol), and mixtures thereof.

In some embodiments, a lignin reaction system comprises: biomass, a solvent, a solid catalyst, one or more reactor vessels, and one or more reaction products. The biomass comprises lignin compounds, and the solvent comprises one or more aliphatic alcohols. The biomass, the solvent, and the solid catalyst are disposed within the one or more reactor vessels, and the one or more reaction products comprise at least one of: 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol), and mixtures thereof.

In some embodiments, a process comprises: contacting a mixture comprising lignin and/or lignin-like molecules with a catalyst to form a reaction mixture, and producing one or more reaction products. The contacting occurs without any addition of molecular hydrogen, and the one or more reaction products are selected from the group consisting of: 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol), and mixtures thereof.

In some embodiments, a process comprises: contacting a mixture comprising lignin and/or lignin-like molecules with a catalyst to form a reaction mixture, producing one or more reaction products, and producing at least one of a phenolic resin or a thermoset from at least a portion of the one or more reaction products. The one or more reaction products are selected from the group consisting of: 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol), and mixtures thereof.

While multiple embodiments are disclosed, still other embodiments will become apparent to those skilled in the art from the following detailed description. As will be apparent, certain some embodiments, as disclosed herein, are capable of modifications in various aspects without departing from the spirit and scope of the claims as presented herein. Accordingly, the detailed description hereinbelow is to be regarded as illustrative in nature and not restrictive.

### BRIEF DESCRIPTION OF THE DRAWINGS

The following figures illustrate embodiments of the subject matter disclosed herein. The claimed subject matter may be understood by reference to the following description taken in conjunction with the accompanying figures, in which:

FIGS. 1A and 1B are formulas for products produced from a lignin extraction and upgrading process according to an embodiment.

FIGS. 2A-2D are additional formulas for products produced from a lignin extraction and upgrading process according to an embodiment.

FIG. 3 is a schematic illustration of a system for the processing of lignin containing biomass according to some embodiments.

FIG. 4 is a schematic illustration of another system for processing of lignin containing biomass according to some embodiments.

FIG. 5 is a schematic flow diagram of a system for processing of biomass according to some embodiments.

FIG. 6A-6D are schematic lignin extraction reactor configurations according to some embodiments.

FIG. 7 is a schematic flow diagram of another system for processing of biomass according to some embodiments.

FIG. 8 illustrates an HPLC/UV-vis chromatogram for the liquid product from Example 1.

FIG. 9 illustrates an HPLC/UV-vis chromatogram for the liquid product from Example 2.

FIG. 10 illustrates an HPLC/UV-vis chromatogram for the liquid product from Example 3.

FIG. 11 illustrates an HPLC/UV-vis chromatogram for the liquid product from Example 4.

FIG. 12 illustrates an HPLC/UV-vis chromatogram for the liquid product from Example 5.

FIG. 13 illustrates an HPLC/UV-vis chromatogram for the liquid product from Example 6.

FIG. 14 illustrates an HPLC/UV-vis chromatogram for the liquid product from Example 7.

FIG. 15 illustrates an HPLC/UV-vis chromatogram for the liquid product from Example 9.

FIG. 16 illustrates the reaction chemistry and formulas for the anticipated reactions indicated in Example 12.

FIG. 17 illustrates the reaction chemistry and formulas for the anticipated reactions indicated in Example 13.

FIG. 18 illustrates the formula for the anticipated product as indicated in Example 14.

FIG. 19 illustrates the formulas for the epoxy polymers described in Example 15.

Various objects, features, aspects and advantages of the present invention will become more apparent from the following detailed description of embodiments of the invention.

### DETAILED DESCRIPTION

It should be understood at the outset that although an illustrative implementation of one or more embodiments are provided below, the disclosed systems and methods may be implemented using any number of techniques, whether currently known or in existence. The disclosure should in no way be limited to the illustrative implementations, drawings, and techniques illustrated hereinbelow, including the exemplary designs and implementations illustrated and described herein, but may be modified within the scope of the appended claims along with their full scope of equivalents. Thus, while multiple embodiments are disclosed, still other embodiments will become apparent to those skilled in the art from the following detailed description. As will be apparent, certain embodiments, as disclosed herein, are capable of modifications in various aspects without departing from the spirit and scope of the claims as presented herein. Accordingly, the detailed description hereinbelow is to be regarded as illustrative in nature and not restrictive.

There is currently a lack of technology to process specific high value aromatic compounds directly from lignin containing biomass. Many processes target the commodity chemicals benzene, toluene, and xylenes (BTX). These processes are demonstrated using model compounds or liquid slurries that contain lignin compounds. Raw biomass (grasses, woods, non-food agricultural byproducts, etc.) that contains lignin is typically pre-treated with methods such as, but not limited to: supercritical carbon dioxide, enzyme hydrolysis, steam explosion, Organosolv, Alcell, pyrolysis, or Kraft pre-treatments to produce separate lignin-rich feed streams and carbohydrate-rich products. The present process has found these pre-treated lignin streams to be less selective, such that dozens of compounds are yielded in near equal amounts making separations difficult and diminishing the value of the process. It has been found that batch conditions using hydrogen gas and a hydrodeoxygenation catalyst containing palladium on carbon can produce a liquid aromatic stream comprised mostly of DHE and DMPP. This process can be used for the production of transportation fuels produced from further treatment of the cellulose residue.

Commercially relevant processes to upgrade the value of lignin require continuous operation. Often these continuous processes utilize continuously stirred reactors (CSTR), bubble reactors, and other designs in which the catalyst and biomass are well mixed. These designs suffer because the catalyst becomes well mixed with the solid residues and product streams. In order to adequately remove metallic catalysts from the product streams the process becomes overly complicated and the number of process units increases.

This present disclosure describes a chemical process for converting lignocellulosic-containing biomass to more valuable chemical products including, but not limited to, dihydroeugenol (DHE, 2-methoxy-4-propylphenol), DMPP (2,6-dimethoxy-4-propylphenol), dihydroconiferyl alcohol (DHE-OH, 4-(3-hydroxypropyl)-2-methoxyphenol), dihydrosinapyl alcohol (DMPP-OH, 4-(3-hydroxypropyl)-2,6-dimethoxyphenol), isoeugenol (2-methoxy-4-(prop-1-en-1-yl)phenol), i-DMPP (2,6-dimethoxy-4-(prop-1-en-1-yl)phenol), ferulate (ferulic acid ester), coumarate (coumaric acid ester), esters of dihydroferulic acid (3-(4-hydroxy-3-methoxyphenyl)propionic acid), esters of phloretic acid (3-(4-hydroxyphenyl)propionic acid), and other related cinnamic acids. The biomass sources include grasses, woods, and agricultural biomass. The process can be carried out as either a batch or continuous operation. In one embodiment of the process, the delignification of the biomass and the catalyzed hydrodeoxygenation of the soluble lignin occur in a single reaction vessel. In other processes the delignification takes place independent of the catalyzed hydrodeoxygenation. Other lignin feed streams include Kraft, Organosolv, or any other lignin-rich biomass supply stream.

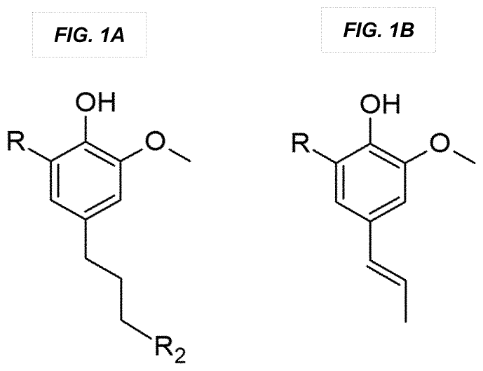

The present process is related to the batch or continuous extraction of specific aromatic high value chemicals from lignin in untreated biomass. Depending on the biomass and reaction conditions used, the process can be tailored to select for DHE, DMPP, DHE-OH, DMPP-OH, isoeugenol, and i-DMPP, FIG. 1. As shown in FIG. 1A, R and R₂ can represent a hydrogen, methoxy group, or —OH group such that: DHE: R═H and R₂═H, DMPP: R═OCH₃ and R₂═H, DHE-OH: R═H and R₂═OH, DMPP-OH: R═OCH₃ and R₂═OH. As shown in FIG. 1B, R can represent a hydrogen or methoxy group such that: isoeugenol: R═H, i-DMPP: R═OCH₃.

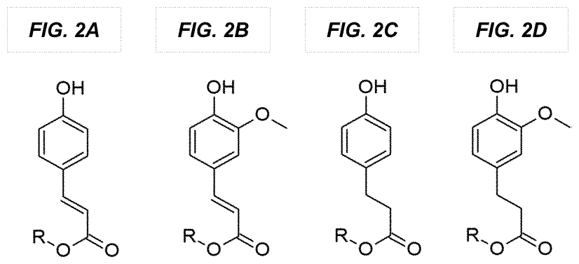

Depending on the biomass and reaction conditions used, the process can also be tailored to produce ferulate (ferulic acid esters of the variety methyl-, ethyl-, propyl-, butyl-, or any variation thereof), coumarate (coumaric acid esters of the variety methyl-, ethyl-, propyl-, butyl, or any variation thereof), dihydroferulic acid esters of the variety methyl-, ethyl-, propyl-, butyl, or any variation thereof, phloretic acid esters of the variety methyl-, ethyl-, propyl-, butyl, or any variation thereof, as shown in FIG. 2. As shown in FIG. 2A, R can represent a hydrogen or alkyl group such that: coumaric acid: R═H, methyl coumarate: R═CH₃, ethyl coumarate: R═C₂H₅, propyl coumarate: R═C₃H₇, butyl coumarate: R═C₄H₉. As shown in FIG. 2B, R can represent a hydrogen or alkyl group such that: ferulic acid: R═H, methyl ferulate: R═CH₃, ethyl ferulate: R═C₂H₅, propyl ferulate: R═C₃H₇, butyl ferulate: R═C₄H₉. As shown in FIG. 2C, R can represent a hydrogen or alkyl group such that: 3-(4-hydroxyphenyl)propionic acid methyl ester: R═CH₃, 3-(4-hydroxyphenyl)propionic acid ethyl ester: R═C₂H₅, 3-(4-hydroxyphenyl)propionic acid propyl ester: R═C₃H₇, 3-(4-hydroxyphenyl)propionic acid butyl ester: R═C₄H₉. As shown in FIG. 2D, R can represent a hydrogen or alkyl group such that: 3-(4-hydroxy-3-methoxyphenyl)propionic acid methyl ester: R═CH₃, 3-(4-hydroxy-3-methoxyphenyl)propionic acid ethyl ester: R═C₂H₅, 3-(4-hydroxy-3-methoxyphenyl)propionic acid propyl ester: R═C₃H₇, 3-(4-hydroxy-3-methoxyphenyl)propionic acid butyl ester: R═C₄H₉. The present process makes use of a hydrodeoxygenation catalyst and the use of hydrogen transfer reagent. Under continuous operation, the biomass and catalytic zones can be separate, which can reduce or eliminate toxic catalyst particles in the product streams. At the end of the process the products are clean, solid cellulose and a liquid stream of lignin products which can be purified by methods such as distillation and precipitation to produce pure high value aromatic compounds and not a crude lignin oil stream. The present process is unique in that the desired compounds can be recovered in relatively large amounts with high purity, and the products can comprise aromatic compounds. Traditional methods for breaking down lignin from cellulose produce crude lignin streams that contain dozens of lignin monomers, dimers, and oligomers which cannot be purified to a high degree with distillation or other traditional separation methods. Often the liquid phase is referred to as technical lignin oil, which has lower value than pure compounds.

The process described herein for producing molecules such as DHE, DMPP, DHE-OH, and DMPP-OH from biomass allows for the produced compounds to be used in making phenolic based resins and thermosets. These molecules can also be combined with co-produced cellulose fibers to make moldable cross-linked networks. For example, wood fiber can be used as a feedstock and the produced aromatic molecules can be combined to make resin or combined subsequently with the co-produced cellulose fiber and cured with amine to make thermoset networks.

Other applications of DHE, DMPP, DHE-OH, and DMPP-OH include derivatization through group protection (methyl, ethyl, propyl, etc.) or trans-esterification to make flavor and fragrance molecules.

Whereas some techniques makes use of added high pressure hydrogen to hydrogenate biomass, the present processes and systems use specific solvents, unique reaction environments, and catalysts to provide for in situ generation and consumption of hydrogen to facilitate the hydrodeoxygenation and conversion of the lignins. This allows the process to operate without the addition of any elemental or free hydrogen into the reaction system.

The biomass used can include any suitable source of biomass containing lignin compounds. In some embodiments, the biomass can comprise lignin containing grasses, agricultural residue, wood, and any combination thereof. In some embodiments, woody biomass can be used based on its high percentage of lignin (e.g., greater than about 10 wt. %, greater than about 15 wt. %, or greater than about 19 wt. %). The reaction parameters can be similar for both batch and continuous operation.

The solvent used in the system can comprise an alcohol (e.g., an aliphatic alcohol, etc.), and optionally, water. In some embodiments, the solvent can comprise between about 50-100% by volume alcohol (e.g., methanol, ethanol, butanols, propanols, or any mixture thereof), and the balance being 0-50% by volume water. In some embodiments, the solvent can comprise between about 60%-100% by volume alcohol with the balance being water. In some embodiments, a mixture of methanol, ethanol, and water can be used to form the solvent.

The hydrogen source for the catalyst can be provided by the solvent. The solid hydrodeoxygenation catalyst can be mixed with the biomass or held independently as to avoid contamination. The hydrodeoxygenation catalyst can comprise a transition metal catalyst usually from groups 7-11, or any mixture thereof. For example, the catalyst can comprise Pt, Pd, Ni, Ru, Rh, Ir, Co, Fe, alloys thereof, oxides thereof, and/or mixtures thereof.

In some embodiments, the catalyst can comprise a nickel sponge catalyst (e.g., Raney nickel). The catalyst can be used in a weight ratio of the catalyst to biomass of between about 1:1,000 to 1:1 (wt/wt). When used, the nickel sponge catalyst can be used alone or contain one or more promoters. The promotors can include various elements including, but not limited to, molybdenum, iron, and/or chromium, and the promoters can be present in any suitable amounts, such as, between about 0.1 to about 20% by mass of the nickel, or between about 1% to 10% by mass of the nickel.

In some embodiments, the catalyst can comprise a solid acid component. Additional use of a solid acid component in the catalyst can affect the selectivity of the reaction. Solid acid catalysts include zeolites, activated carbon, ammonium tungstate, etc. When present, the solid acid component can be present in an amount between about 1:10-10:1 (wt/wt) ratio of the solid acid component to the transition metal catalyst component. In some embodiments, the catalyst can comprise a solid acid component.

In some embodiments, promotors can be used with catalyst portions other than the nickel sponge. For example, promotors can be present in the hydrodeoxygenation catalyst comprising a transition metal catalyst from groups 7-11 and/or the solid acid component. The promotors can comprise molybdenum, iron, and/or chromium, and can be present in an amount of between about 0.1% to about 20% by weight of the catalyst.

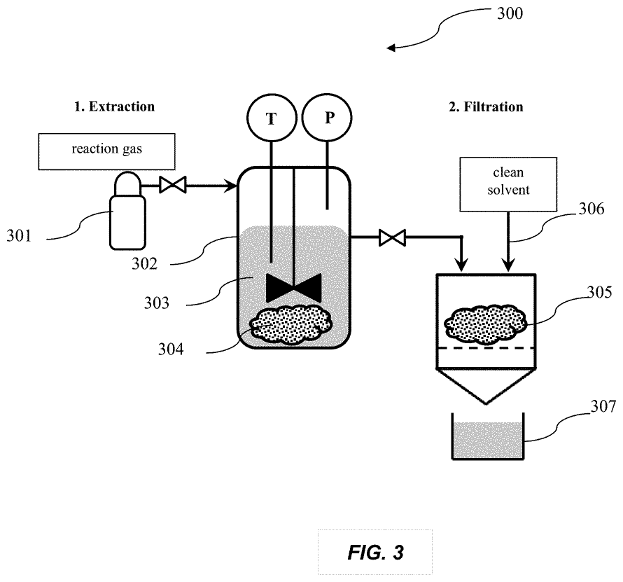

The extraction of high value aromatic chemicals from biomass can be performed using a variety of reactor configurations. In an embodiment, a stirred batch reactor configuration 300 is shown in FIG. 3. If it is desirable to prevent mixing of the solid catalyst (e.g., in powder or slid form) and the biomass, the catalyst can be packed into a porous cage or package. If the catalyst mixing with the products is not an issue, the materials can all be mixed directly into the reaction vessel 302, or pumped into the reaction vessel 302 as slurry of biomass, catalyst, and co-catalyst 304 in solvent. For use in a single reactor vessel, the mass to mass ratio for solvent:biomass can be in the range of between about 2-20. In some embodiments, a mass to mass ratio for solvent:biomass of between about 11-16 can be used. The solvent 303 can comprise any of the solvent mixtures described herein. In some embodiments, the solvent can comprise between about 50-100% by volume alcohols (methanol, ethanol, propanols, butanols, or any mixture thereof) and 0-50% by volume water. The water concentration can change the total yield of aromatic compounds and/or the selectivity for individual compounds. The mass to mass ratio for biomass:transition metal catalyst can be in the range of between about 1-1000, between about 2-100, or between about 5-10. The mass to mass ratio for transition metal catalyst:solid acid catalyst can be in the range of between about 0.1-5, or between about 0.3-3. After loading all the starting materials into the reactor 302, the reactor 302 can be sealed shut. The reaction atmosphere can be purged with an inert atmosphere 301 such as nitrogen or argon. The reaction atmosphere can then be pressurized to between about 1-35 bar at room temperature. The reactor can be heated at a rate of between about 100-300° C. hr⁻¹ (e.g., about 300° C. hr⁻¹) to a dwell temperature of between about 140-250° C. The reactor can maintain the maximum extraction temperature for between 1-15 hours, with between about 2-12 being standard. A stirring mechanism can be used, and can be operated between about 100-600 rpm, between about from about 150 to 250 rpm, or at about 200 rpm.

After the reaction is carried out and the reactor cooled, the contents of the reaction vessel 302 can be removed by pumping or gravity. The post reaction slurry 305, which can contain cellulose, hemicellulose, catalyst, and co-catalyst, can be filtered from the solvent, which can contain aromatic products 307 such as dihydroeugenol (DHE, 2-methoxy-4-propylphenol), DMPP (2,6-dimethoxy-4-propylphenol), dihydroconiferyl alcohol (DHE-OH, 4-(3-hydroxypropyl)-2-methoxyphenol), dihydrosinapyl alcohol (DMPP-OH, 4-(3-hydroxypropyl)-2,6-dimethoxyphenol), isoeugenol (2-methoxy-4-(prop-1-en-1-yl)phenol), i-DMPP (2,6-dimethoxy-4-(prop-1-en-1-yl)phenol), ferulate (ferulic acid ester), coumarate (coumaric acid ester), esters of dihydroferulic acid (3-(4-hydroxy-3-methoxyphenyl)propionic acid), esters of phloretic acid (3-(4-hydroxyphenyl)propionic acid), and/or other related cinnamic acids. The solid residue 305 can be rinsed with clean solvent 306 using 50-150% the volume of the filtered solids. The wet solids can then be pressed to further remove solvent captured in the solids.

In some embodiments, the yields of targeted aromatic phenols (DHE, DMPP, DHE-OH, DMPP-OH, and/or isoeugenol) can be achieved using a Ni catalyst and activated carbon. Activated carbon can increase the yield of the monomers. The states of the Ni catalyst used (e.g., the use of NiO₂ or Ni(0)) may not have a significant effect on the combined yield in the presence of activated carbon. The propyl aromatics (DHE and DMPP) are selected for when the solution is 100% alcohol. If an inert atmosphere is used, the reaction produces less alcohol containing aromatics (DHE-OH and DMPP-OH). For reaction conditions utilizing 1-50% water, shorter reaction times can be used and the selectivity switches to favor alcohol containing aromatics (DHE-OH and DMPP-OH) over the terminating propyl aromatics (DHE and DMPP). The lignin content of the biomass can also change the selectivity of DHE based products vs DMPP products. For example, pine does not contain S-type lignin necessary to produce DMPP. Genetically modified woods can also be used to produce a single monomer in large quantities.

As demonstrated by the embodiment of FIG. 3, lignin can be extracted and upgraded to aromatic molecules directly from biomass without supplying external hydrogen. The process can occur in a single reaction vessel in some embodiments such that the resulting product stream can comprise upgraded aromatic compounds suitable for a variety of downstream uses.

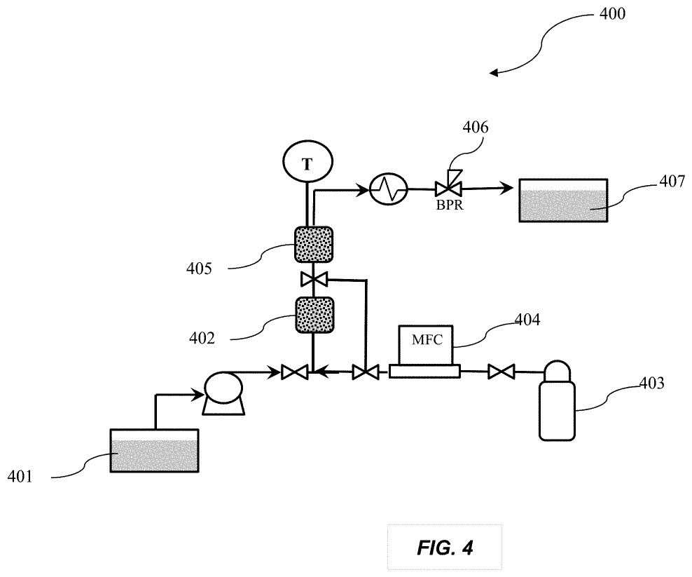

A continuous method to extract aromatic monomers from lignin containing biomass can be achieved in a continuous reactor such as a continuous stirred tank reactor, a packed bed reactor, a screw extruder, and the like. As shown in FIG. 4, a packed bed reactor can be used in a reactor system 400 some embodiments. The solvent 401 (e.g., comprising 50-100% alcohol in a balance of water) can be pumped into a first bed 402 containing biomass. The biomass can be loaded as a stationary bed 402 upstream of a second packed bed 405 containing the catalyst. Both packed beds can be independently heated, and the heated and pressurized solvent can be insulated as it flows between each bed. High pressure reaction gas 403 can be introduced (as necessary) to the reactor at a controlled rate by a mass flow controller (MFC) 404. Valves can be used to introduce the gas before or after the biomass bed 402 to allow the catalyst to be pretreated. A back pressure regulator (BPR) 406 can be used downstream of the packed beds to maintain the reactor at a steady pressure, typically between about 5-55 bar. The typical operating conditions include bed temperatures (e.g., for both beds) at between about 150-250° C., a system pressure of between about 5-55 bar, a catalyst residence time of between about 0.25-1 hour for the solvent, and an optional gas flow. The extraction solvent and reaction gas exit the heated reaction zone and can be cooled by a heat exchanger. The reaction solvent which contains the desired aromatic products 407 can be collected in a reservoir at the exit of the reactor after cooling the solvent such that it is a liquid. The desired aromatics (e.g., dihydroeugenol (DHE, 2-methoxy-4-propylphenol), DMPP (2,6-dimethoxy-4-propylphenol), dihydroconiferyl alcohol (DHE-OH, 4-(3-hydroxypropyl)-2-methoxyphenol), dihydrosinapyl alcohol (DMPP-OH, 4-(3-hydroxypropyl)-2,6-dimethoxyphenol), isoeugenol (2-methoxy-4-(prop-1-en-1-yl)phenol), i-DMPP (2,6-dimethoxy-4-(prop-1-en-1-yl)phenol), ferulate (ferulic acid ester), coumarate (coumaric acid ester), esters of dihydroferulic acid (3-(4-hydroxy-3-methoxyphenyl)propionic acid), esters of phloretic acid (3-(4-hydroxyphenyl)propionic acid), and/or other related cinnamic acids) are contained in the liquid collection reservoir and the solid biomass can be clean cellulose.

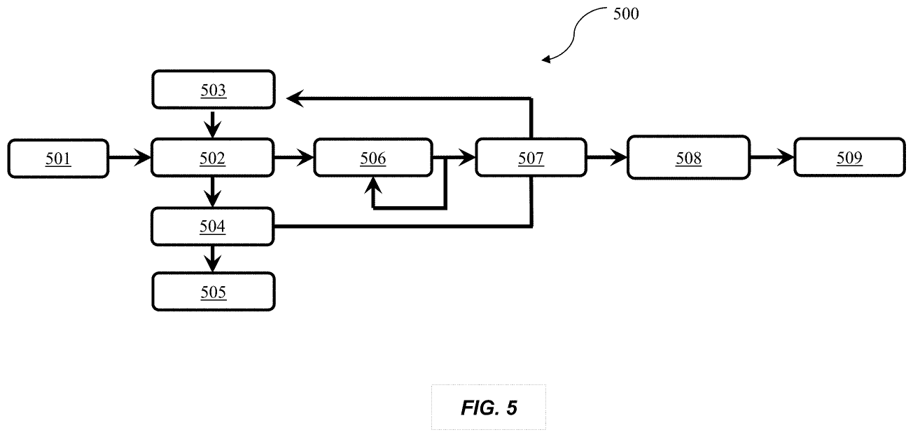

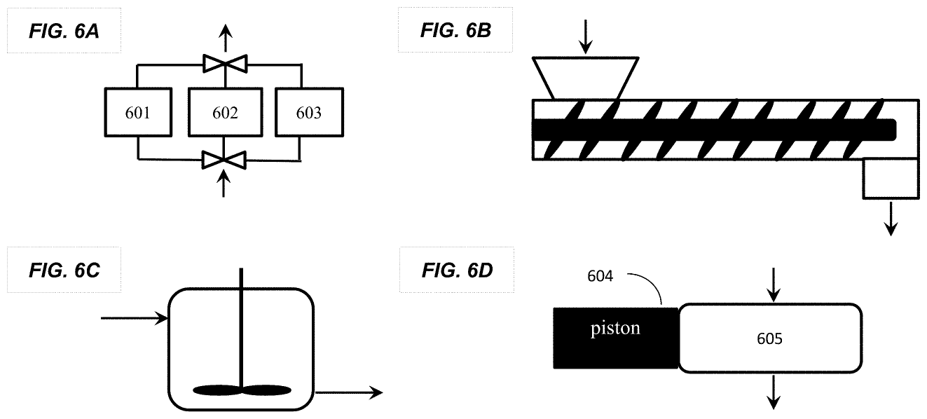

FIGS. 5-7 show process schematics for the scaled up extraction. FIG. 5 shows a commercial process flow chart for the extraction of valuable chemical products that can include, but are not limited to, dihydroeugenol (DHE, 2-methoxy-4-propylphenol), DMPP (2,6-dimethoxy-4-propylphenol), dihydroconiferyl alcohol (DHE-OH, 4-(3-hydroxypropyl)-2-methoxyphenol), dihydrosinapyl alcohol (DMPP-OH, 4-(3-hydroxypropyl)-2,6-dimethoxyphenol), isoeugenol (2-methoxy-4-(prop-1-en-1-yl)phenol), i-DMPP (2,6-dimethoxy-4-(prop-1-en-1-yl)phenol), ferulate (ferulic acid ester), coumarate (coumaric acid ester), esters of dihydroferulic acid (3-(4-hydroxy-3-methoxyphenyl)propionic acid), esters of phloretic acid (3-(4-hydroxyphenyl)propionic acid), and/or other related cinnamic acids. FIG. 5 illustrates a process in which milled biomass 501 can be fed into a lignin extractor 502. The lignin extractor 502 can be set as a semi-batch or continuous process, where possible configurations are shown in FIG. 6. During operation the reactor temperatures can be ramped up at a rate of between about 100-300° C. hr⁻¹ to the set bed temperatures. The reactor 502 can be operated such that the biomass is contacted with the solvent for between about 4-8 hours at the reaction temperature. After appropriate extraction time, the reactor 502 can be cooled, for example to room temperature, depressurized to atmospheric pressure, and the spent solid biomass can be removed. Within FIG. 5, a solvent (e.g., between about 50-100% alcohol in water) 503 can be fed into the lignin extractor 502. A dryer 504 (e.g., a centrifuge dryer, etc.) can be used to remove the solid carbohydrate byproduct 505. Solvent extracted lignin can be pumped from the lignin extractor 502 to the catalyst and co-catalyst container 506 for reaction. Solvent can be recycled back into the catalyst and co-catalyst container 506 to increase yields of desired products from the extracted lignin. The solvent can be recovered using a solvent recovery system 507 and purified for further lignin extractions. The products can be optionally separated using a product separation system 508 such as distillation and other optional separation techniques. Aromatic products 509 from lignin can be obtained from the separation system.

FIGS. 6A-6D illustrated possible configurations for a lignin extractor, such as the lignin extractor 502 described with respect to FIG. 5. For continuous operation, multiple reactor designs are possible, such as those shown in FIGS. 6A-6D. In FIG. 6A, a plurality of biomass packed beds 601, 602, 603 can be operated in parallel, in which one or more reactors are operating at temperature while the non-operating reactor(s) are in the process of start-up or shut-down. While three beds are shown in FIG. 6A, any number of reactors can be used to provide the throughput capacity as desired. Further, any suitable valving configurations can be used to selectively isolate one or more reactors while allowing others to operate during warm-up, reaction, or shut-down.

In some embodiments, one or more of the packed beds containing biomass can be replaced with a heated screw extruder that can function continuously, such as schematically illustrated in FIG. 6B. As shown, biomass can be fed continuously into the screw extruder where it is held in contact with solvent at the desired temperature of between about 150-250° C. for between about 2-8 hours. Solvent flow can be concurrent or counter-current to the biomass in the screw extruder. The solids can be removed from the extruder while the liquids are then sent to the catalytic bed. The lignin extractor can also be operated as a continuous flow stirred tank reactor (CSTR) with a constant flow of biomass slurry, such as in FIG. 6C. FIG. 6D illustrates another reactor configuration which could operate as a batch or semi-batch process in which a piston 604 compresses the biomass and solvent in the lignin extractor 605 to increase the pressure and temperature. Following reaction, the biomass can be compressed and easily removed.

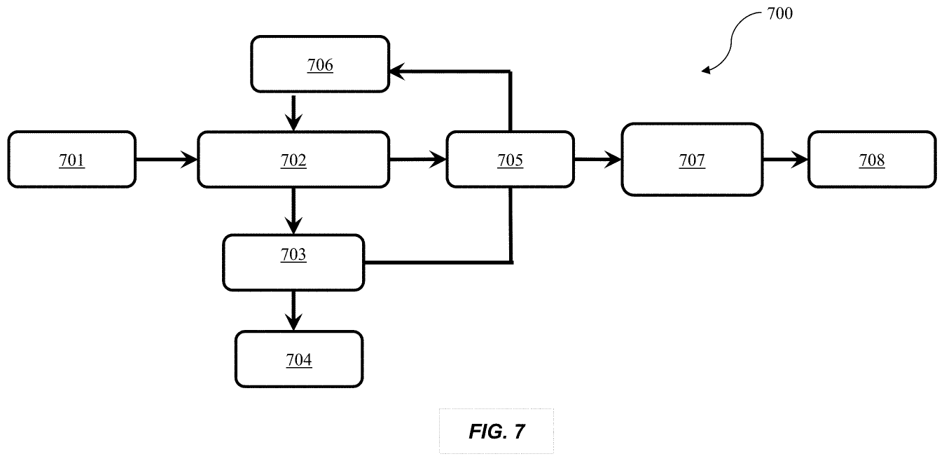

FIG. 7 illustrates a process in which the lignin extraction process and catalytic process are contained in a single operating unit and can be operated as either a semi-batch or continuous process. As shown in FIG. 7, milled biomass 701 can be fed into a lignin extraction and catalysis chamber 702, where the milled biomass 701 can be contacted with a solvent 706 (e.g., between about 50-100% alcohol in water) and a mixture of catalyst and co-catalyst. A dryer 703 (e.g., a centrifuge dryer, etc.) can be used to remove a mixture of solid carbohydrates, catalyst, and co-catalyst 704. A solvent recovery system 705 can be used to return and purify solvent for further lignin extraction. An optional product purification system 707 can use distillation, nanofiltration, and other optional separation techniques to purify the desired aromatic products from lignin. Aromatic products from lignin 708 can be collected at the end of the process.

The aromatic monomers (DHE, DMPP, DHE-OH, DMPP-OH, isoeugenol, and i-DMPP) in the liquid product streams produced by the processes and methods described herein can be separated and purified. For example, an evaporator (flash distillation, rotary evaporator) can be used to optionally concentrate the solution. Vacuum distillation and/or nanofiltration can be used to purify DHE and DMPP. During vacuum distillation the vacuum can be in the range of 10⁻³-10⁻⁵ torr. The column can be operated between 90-200° C. DHE distills at ca. 100° C. and DMPP at ca. 180° C. DHE and DMPP can achieve mass purities of 85-99 wt % by simple short path distillation. Multi tray distillation can achieve mass purity of 95-99 wt %. Thus, relatively pure products can be obtained from the product solutions produced.

As illustrated with respect to FIGS. 4-7, lignin can be extracted from biomass in a lignin extraction reactor. The resulting lignin containing stream can then be used to upgrade the lignin without the addition of an external hydrogen stream or feed to produce aromatic compounds. This process demonstrates the ability to provide hydrogen free lignin upgrading using a lignin containing stream as the starting material. Further, the resulting products can be substantially catalyst free when used with the proper operating conditions.

Using the aromatic products produced by the processes and systems described herein, various additional processes can be used to form additional products. In some embodiments, the product molecules can be used to make thermoset materials. In some embodiments, the reaction products can be reacted to form an epoxy monomer and/or undergo one or more additional reactions to form such an epoxy monomer that can then be reacted with an initiator to form a higher molecular weight product, such as a thermoset material. For example, one or more of the aromatic products can be reacted to form an epoxy monomer. For example, DMPP-OH and/or DHE-OH can be reacted with epihydrochlorin (ECH) to form the epoxy monomer DGEDMPP-OH and/or DGEDHE-OH, respectively. As another example, DMPP-OH and DHE-OH monomers can be reacted with an excess of an excess of epihydrochlorin (ECH) to produce GEDHE-OH and GEDMPP-OH epoxy resins with terminal glycidylated groups

In some embodiments, the reaction products can be reacted to form epoxy precursors. For example, DMPP-OH can be ortho-demethylated using hydrobromic acid (HBr) to form DMPPO-OH. The resulting DMPPO-OH can then be reacted with epihydrochlorin (ECH) to form the epoxy monomer TGEDMPPO-OH, with some benzodioxane monomer formed as well. Similarly, DHE-OH can be ortho-demethylated using HBr to form DHEO-OH. The resulting DHEO-OH can then be reacted with epihydrochlorin (ECH) to form the epoxy monomer TGEDHEO-OH, with some benzodioxane monomer formed as well.

The resulting epoxy monomers can be reacted to form an epoxy polymer. For example, the epoxy monomers DGEDHE-OH and/or DGEDMPP-OH can be reacted with diethylenetriamine (DETA) or a similar amine to form a crosslinked epoxy polymer. The resulting epoxy polymers can be used for a variety of uses. In some embodiments, the epoxy polymers can be used to form a composition material comprising a particulate or fiber phase and a matrix phase. Thus, additional products can be formed using the products of the lignin extraction and upgrading, including renewable polymer materials.

### EXAMPLES

The embodiments having been generally described, the following examples are given as particular embodiments of the disclosure and to demonstrate the practice and advantages thereof. It is understood that the examples are given by way of illustration and are not intended to limit the specification or the claims in any manner.

Within the examples provided herein, the liquid product streams were analyzed with a HPLC equipped with a UV-vis detector. The column was a Zorbax SB-Phenyl reversed-phase C18 HPLC column. The DHE, DMPP, DHE-OH, DMPP-OH, isoeugenol, and i-DMPP in the product stream were quantified using a gradient of water and acetonitrile to elute the products at 30° C. and a flow rate of 0.5 mL min⁻¹. Standard curves for each expected product were created to determine the concentration and yield of each product. Isoeugenol and DHE related compounds were analyzed at 282.8 nm and DMPP related compounds were analyzed at 276.8 nm. The yield of each monomer was based on the lignin content of the biomass used. The lignin composition of each biomass was determined using the acetyl bromide-soluble lignin (ABSL) analysis method.

### Example 1

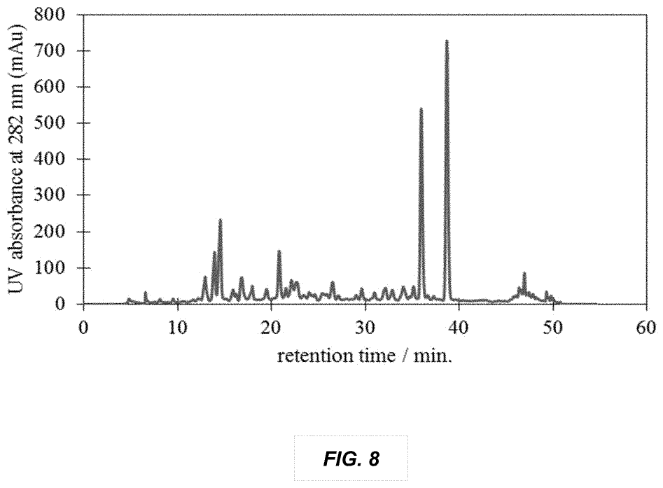

Extraction of aromatics from poplar using a nickel on activated carbon catalyst. In this Example 1, 1 g of 40 mesh wild type 717 poplar (19 wt % lignin) was loaded into a 100 mL stirred batch reactor (Parr Instruments 2430) with 20 mL of methanol. The catalyst was a 10 wt % Ni/C powder catalyst with a mass of 100 mg. The reactor was sealed and purged with 99.999% hydrogen. The reactor was pressurized to ca. 35 bar at room temperature. The temperature was increased to 220° C. at a ramp rate of 300° C. h⁻¹ while stirring at 200 rpm. The dwell time at 220° C. was 12 hours. After cooling to room temperature the solids and liquids were filtered and washed with methanol; producing a total liquid volume of 50 mL. The methanol was analyzed by HPLC/UV-vis, as shown in FIG. 8. DHE, DMPP, DHE-OH, and DMPP-OH eluted at ca. 39 min, 36 min, 15 min, and 14 min, respectively. The yield of desired aromatics based on the lignin content of wild type poplar was 46.4% (Y_{DHE}=9.3%, Y_{DHE-OH}=2.8%, Y_{DMPP}=25.3%, Y_{DMPP-OH}=8.7%, Y_{isoeugenol}=0.4%).

### Example 2

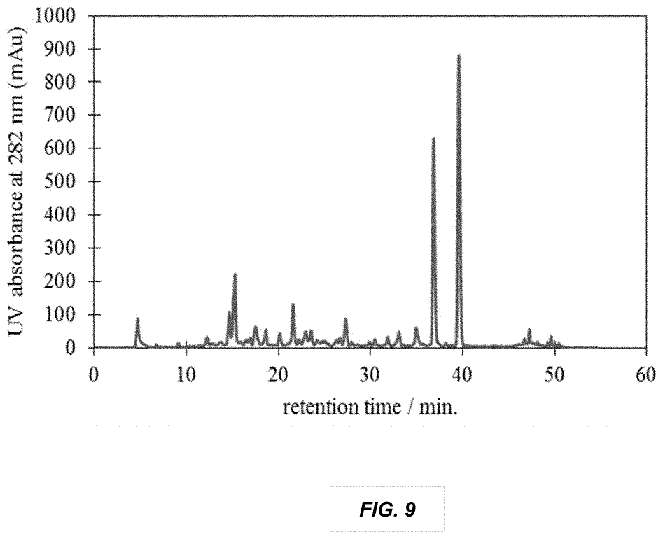

Extraction of aromatics from poplar using a sponge nickel catalyst and activated carbon. In this Example 2, 1 g of 40 mesh wild type 717 poplar (19 wt % lignin) was loaded into a 100 mL stirred batch reactor (Parr Instruments 2430) with 20 mL of methanol. The catalyst was 100 mg of sponge nickel and 200 mg of activated carbon. The reactor was sealed and purged with 99.999% hydrogen. The reactor was pressurized to ca. 35 bar at room temperature. The temperature was increased to 220° C. at a ramp rate of 300° C. h⁻¹ while stirring at 200 rpm. The dwell time at 220° C. was 12 hours. After cooling to room temperature the solids and liquids were filtered and washed with methanol; producing a total liquid volume of 50 mL. The methanol was analyzed by HPLC/UV-vis, as shown in FIG. 9. DHE, DMPP, DHE-OH, and DMPP-OH eluted at ca. 40 min, 37 min, 15 min, and 14 min, respectively. The yield of desired aromatics based on the lignin content of wild type poplar was 50.2% (Y_{DHE}=11.2%, Y_{DHE-OH}=2.6%, Y_{DMPP}=30.9%, Y_{DMPP-OH}=5.6%, Y_{isoeugenol}=not detected), which was the highest total combined yield achieved.

### Example 3

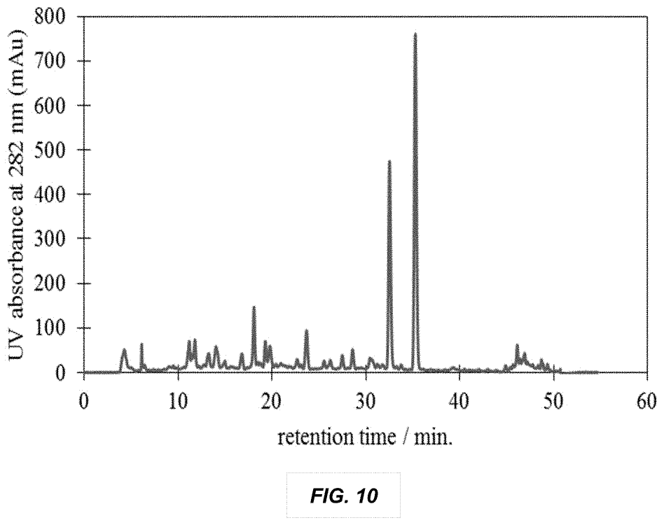

Selective extraction of propyl aromatics from poplar using a sponge nickel catalyst and activated carbon. 1 g of 40 mesh wild type 717 poplar (19 wt % lignin) was loaded into a 100 mL stirred batch reactor (Parr Instruments 2430) with 20 mL of methanol. The catalyst was 100 mg of sponge nickel and 200 mg of activated carbon. The reactor was sealed and purged with 99.999% argon. The reactor was pressurized to ca. 35 bar at room temperature. The temperature was increased to 220° C. at a ramp rate of 300° C. h⁻¹ while stirring at 200 rpm. The dwell time at 220° C. was 12 hours. After cooling to room temperature the solids and liquids were filtered and washed with methanol; producing a total liquid volume of 50 mL. The methanol was analyzed by HPLC/UV-vis, as shown in FIG. 10. DHE, DMPP, DHE-OH, and DMPP-OH eluted at ca. 36 min, 33 min, 12 min, and 11 min, respectively. The yield of desired aromatics based on the lignin content of wild type poplar was 35.9% (Y_{DHE}=9.5%, Y_{DHE-OH}=0.8%, Y_{DMPP}=22.2%, Y_{DMPP-OH}=3.3%, Y_{isoeugenol}=not detected), which was the highest ratio of DHE and DMPP compared to DHE-OH and DMPP-OH.

### Example 4

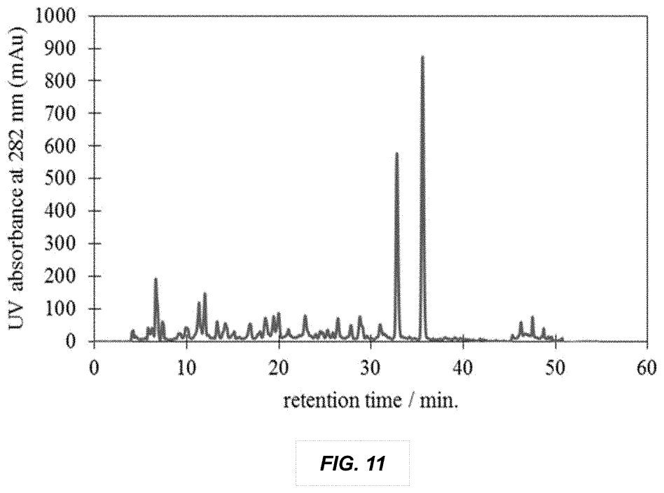

Extraction of aromatics from poplar using a sponge nickel catalyst and activated carbon in ethanol. 1 g of 40 mesh wild type 717 poplar (19 wt % lignin) was loaded into a 100 mL stirred batch reactor (Parr Instruments 2430) with 18 mL of ethanol and 2 mL of water. The catalyst was 200 mg of sponge nickel and 200 mg of activated carbon. The reactor was sealed and purged with 99.999% argon. The reactor was pressurized to ca. 3 bar at room temperature. The temperature was increased to 220° C. at a ramp rate of 300° C. h⁻¹ while stirring at 200 rpm. The dwell time at 220° C. was 12 hours. After cooling to room temperature the solids and liquids were filtered and washed with ethanol; producing a total liquid volume of 50 mL. The solution was analyzed by HPLC/UV-vis, as shown in FIG. 11. DHE, DMPP, DHE-OH, and DMPP-OH eluted at ca. 36 min, 33 min, 12 min, and 11 min, respectively. The yield of desired aromatics based on the lignin content of wild type poplar was 46.1% (Y_{DHE}=10.9% Y_{DHE-OH}=1.4%, Y_{DMPP}=27.4%, Y_{DMPP-OH}=6.5%, Y_{isoeugenol}=not detected).

### Example 5

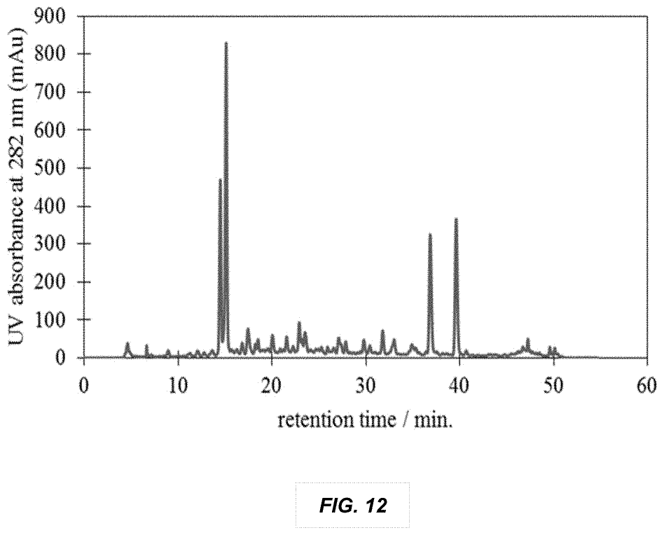

Selective extraction of propanol aromatics from poplar using a sponge nickel catalyst and activated carbon. 1 g of 40 mesh wild type 717 poplar (19 wt % lignin) was loaded into a 100 mL stirred batch reactor (Parr Instruments 2430) with 14 mL of methanol and 6 mL of water. The catalyst was 100 mg of sponge nickel and 100 mg of activated carbon. The reactor was sealed and purged with 99.999% hydrogen. The reactor was pressurized to ca. 35 bar at room temperature. The temperature was increased to 220° C. at a ramp rate of 300° C. h⁻¹ while stirring at 200 rpm. The dwell time at 220° C. was 3 hours. After cooling to room temperature the solids and liquids were filtered and washed with methanol; producing a total liquid volume of 50 mL. The methanol was analyzed by HPLC/UV-vis, as shown in FIG. 12. DHE, DMPP, DHE-OH, and DMPP-OH eluted at ca. 40 min, 37 min, 15 min, and 14 min, respectively. The yield of desired aromatics based on the lignin content of wild type poplar was 48.7% (Y_{DHE}=4.6%, Y_{DHE-OH}=8.4%, Y_{DMPP}=14.7%, Y_{DMPP-OH}=21.0%, Y_{isoeugenol}=not detected), which was the highest ratio of DHE-OH and DMPP-OH compared to DHE and DMPP.

### Example 6

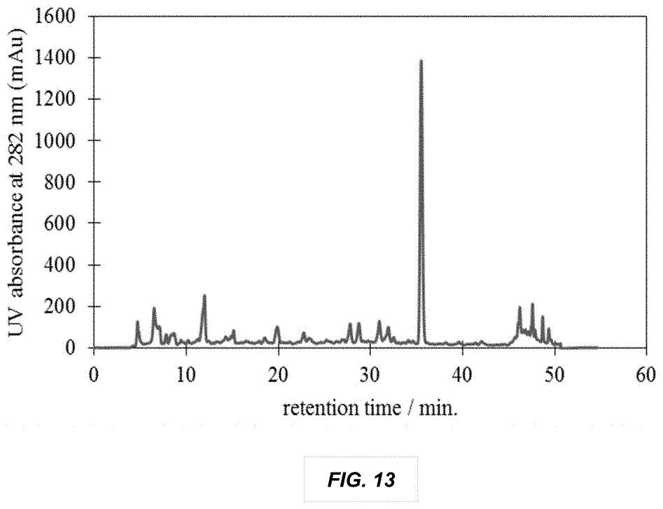

Selective extraction of propyl aromatics from pine using a sponge nickel catalyst and activated carbon. 1 g of pine crumbles (31 wt % lignin) was loaded into a 100 mL stirred batch reactor (Parr Instruments 2430) with 18 mL of ethanol and 2 mL water. The catalyst was 200 mg of sponge nickel and 200 mg of activated carbon. The reactor was sealed and purged with 99.999% argon. The reactor was pressurized to ca. 3 bar at room temperature. The temperature was increased to 220° C. at a ramp rate of 300° C. h¹ while stirring at 200 rpm. The dwell time at 220° C. was 12 hours. After cooling to room temperature the solids and liquids were filtered and washed with ethanol; producing a total liquid volume of 50 mL. The solution was analyzed by HPLC/UV-vis, as shown in FIG. 13. DHE and DHE-OH eluted at ca. 36 min and 12 min, respectively. The yield of desired aromatics based on the lignin content pine was 14.0% (Y_{DHE}=11.3%, Y_{DDE-OH}=2.2%, Y_{DMPP}=not detected, Y_{DMPP-OH}=not detected, Y_{isoeugenol}=not detected). DMPP was not detected because pine does not contain the S-type lignin required for DMPP extraction.

### Example 7

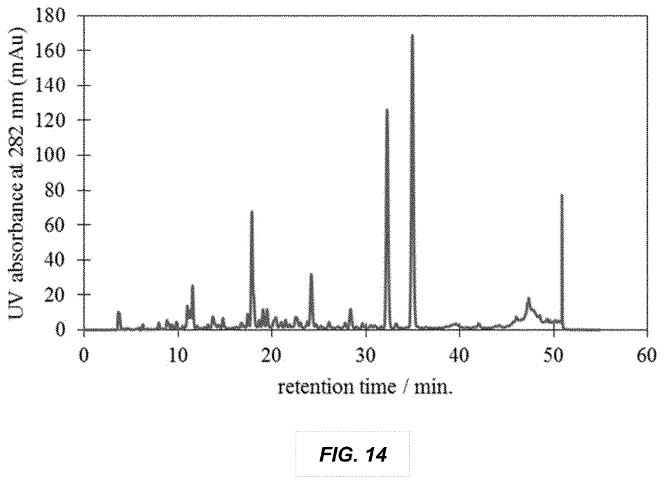

Continuous extraction of aromatics in a packed bed reactor. 10.04 g of 40 mesh wild type 717 poplar (19 wt % lignin) was loaded into a 90 mL packed bed reactor, located inside an insulated furnace. Downstream of the wood biomass, a second packed bed was composed of 5 g Ni sponge catalyst and 8 g activated carbon. The back pressure regulator was set to 7 bar, and the reactor was filled with methanol at a flow rate of 1.5 mL min⁻¹, which gave a residence time of ca. 60 minutes over the catalytic bed. The furnace was ramped to a temperature of 220° C. while the back pressure regulator was adjusted to 62 bar. After the internal temperature reached 220° C., hydrogen was introduced to the reactor at a molar ratio of 205 mol methanol:mol hydrogen. The reactor was operated for 7 hours at temperature. The solution was analyzed by HPLC/UV-vis, as shown in FIG. 14. DHE, DMPP, DHE-OH, and DMPP-OH eluted at ca. 35 min, 32 min, 12 min, and 11 min, respectively. The yield of desired aromatics based on the lignin content of the wild type poplar was 30.4% (Y_{DHE}=7.6%, Y_{DHE-OH}=0.9%, Y_{DMPP}=20%, Y_{DMPP-OH}=1.9%, Y_{isoeugenol}=not detected). The mass loss of the poplar was ca. 30 wt %.

### Example 8

Short path distillation of DHE and DMPP. Post extraction solutions of methanol containing DHE and DMPP were distilled using a short path distillation column. The aromatic products were placed in the still pot which was heated with an oil bath. The system was under a vacuum of ca. 10⁻³-10⁻⁴ torr. DHE distilled at a bath temperature of 100° C. and an internal temperature of 49° C. in the column. DMPP distilled at an external bath temperature of 180° C. and an internal temperature of 90° C. in the column. The DHE fraction was 91.5 wt % pure and the DMPP fraction was 85.3 wt % pure, with the main impurity being DHE.

### Example 9

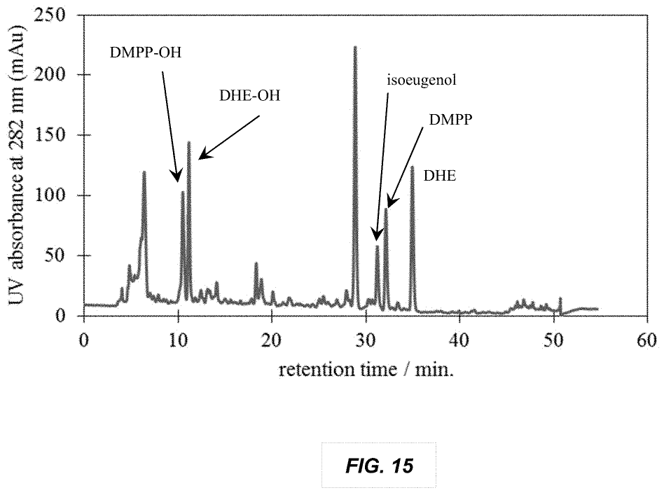

Continuous extraction of aromatics in a packed bed reactor with no hydrogen. 10.15 g of 40 mesh wild type 717 poplar (19 wt % lignin) was loaded into a packed bed reactor with an inner diameter of ⅞ inches and a bed length of 3 inches. Downstream of the wood biomass was a packed bed of raney nickel (2 g) and activated carbon (2 g). The internal temperature of the biomass and catalyst was maintained at 210° C. A back pressure regulator maintained a pressure of 45 bar. 90% ethanol in water was pumped through the reactor at a flow rate of 1.5 mL min⁻¹. After 7 hours of reaction the temperature and pressure were returned to ambient conditions. The solution was analyzed by HPLC/UV-vis, as shown in FIG. 15. DHE, DMPP, isoeugenol, DHE-OH, and DMPP-OH eluted at ca. 35 min, 32 min, 31 min, 11 min, and 10 min, respectfully. The yield of desired aromatics based on the lignin content of the wild type poplar was 40.8% (Y_{DHE}=5.1%, Y_{DHE-OH}=4.2%, Y_{DMPP}=12.4%, Y_{DMPP-OH}=17.6%, Y_{isoeugenol}=1.6%). The mass loss of the poplar was ca. 34 wt %.

### Example 10

Continuous extraction in a series of packed bed reactors. In a prophetic example, 10 g of 40 mesh wild type 717 poplar (19 wt % lignin) is loaded into a packed bed reactor located upstream of a second packed bed reactor. The catalyst (2 g raney nickel and 2 g activated carbon) is loaded in the downstream reactor. The internal temperature of the biomass bed is held at a 180° C., which is lower than the catalytic zone. The catalyst is maintained at 210° C. The pressure of the entire system is maintained at ca. 45 bar. 90% ethanol in water is pumped through the reactor at a flow rate of 1.5 mL min⁻¹ for 7 hours. The yield of desired aromatics is 40.8% (Y_{DHE}=5.1%, Y_{DHE-OH}=4.2%, Y_{DMPP}=12.4%, Y_{DMPP-OH}=17.6%, Y_{isoeugenol}=1.6%). The mass loss of the poplar is ca. 34 wt %.

### Example 11

Continuous extraction in series with a CSTR and PBR. In another prophetic example, 10 g of 40 mesh wild type 717 poplar (19 wt % lignin) is loaded into a CSTR. The CSTR is operated at 180° C. with an inert atmosphere. The extraction solvent is 90% ethanol. After 3 hours of reaction the solvent is removed from the reactor while still hot and pumped over a catalytic bed in a packed bed reactor. The catalyst is raney nickel (2 g) and activated carbon (2 g). The catalyst is maintained at 210° C. The pressure of the PBR is maintained at ca. 45 bar. The yield of desired aromatics is 40.8% (Y_{DHE}=5.1%, Y_{DHE-OH}=4.2%, Y_{DMPP}=12.4%, Y_{DMPP-OH}=17.6%, Y_{isoeugenol}=1.6%). The mass loss of the poplar was ca. 34 wt %.

### Example 12

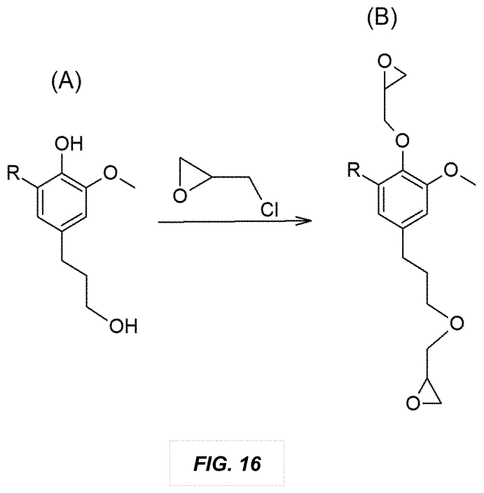

Epoxidation of DMPP-OH and DHE-OH with epihydrochlorin to form epoxy monomers. In another prophetic example, DMPP-OH is reacted with epihydrochlorin (ECH) to form the epoxy monomer DGEDMPP-OH. A schematic representation of the anticipated reaction of DMPP with ECH to form DGEDMPP-OH is shown in FIG. 16. In a similar fashion, DHE-OH can be reacted with epihydrochlorin (ECH) to form the epoxy monomer DGEDHE-OH. As shown in FIG. 16, in formula A: R can represent a hydrogen or methoxy group such that the starting material to be reacted with ECH is one of the following: DMPP-OH:R═OCH₃, DHE-OH:R═H. As shown in FIG. 16, in formula B, R can represent a hydrogen or methoxy group such that the reaction product is one of the following: DGEDMPP-OH:R═OCH₃, DGEDHE-OH:R═H.

### Example 13

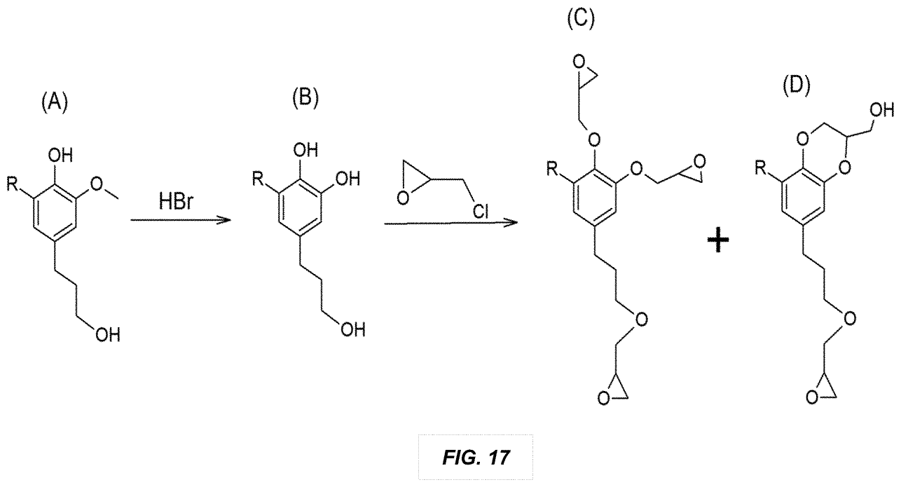

Demethylation and epoxidation of DMPP-OH and DHE-OH to form epoxy monomers. In another prophetic example, DMPP-OH is ortho-demethylated using hydrobromic acid (HBr) to form DMPPO-OH. The resulting DMPPO-OH is then reacted with epihydrochlorin (ECH) to form the epoxy monomer TGEDMPPO-OH, with some benzodioxane monomer formed as well. A schematic representation of the ortho-demethylation of DMPP with HBr to form DMPPO-OH, followed by the reaction of DMPPO-OH with ECH to form TGEDMPPO-OH and the corresponding DMPP-benzodioxane monomer is shown in FIG. 17. n a similar fashion, DHE-OH is ortho-demethylated using HBr to form DHEO-OH. The resulting DHEO-OH is then reacted with epihydrochlorin (ECH) to form the epoxy monomer TGEDHEO-OH, with some benzodioxane monomer formed as well. A schematic representation of the ortho-demethylation of DHE-OH with HBr to form DHEO-OH, followed by the reaction of DHEO-OH with ECH to form TGEDHEO-OH and the corresponding DHE-benzodioxane monomer is shown in FIG. 17. As shown in FIG. 17, in formula A, R can represent a hydrogen or methoxy group such that the starting material to be reacted with HBr is one of the following: DMPP-OH:R═OCH₃, DHE-OH:R═H. In formula B, R can represent a hydrogen or methoxy group such that the intermediate species to be reacted with ECH is one of the following: DMPPO-OH:R═OCH₃, DHEO-OH:R═H. In formula C, R can represent a hydrogen or methoxy group such that a reaction product is one of the following: TGEDMPPO-OH:R═OCH₃, TGEDHEO-OH:R═H. In formula D, R can represent a hydrogen or methoxy group such that a reaction product is one of the following: DMPP-benzodioxane monomer:R═OCH₃, DHE-benzodioxane monomer:R═H.

### Example 14

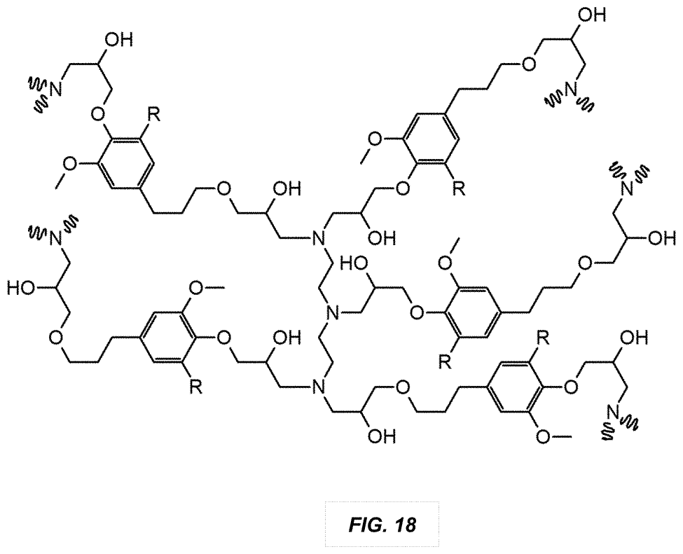

Synthesis of epoxy polymers. In another prophetic example, epoxy monomers DGEDHE-OH or DGEDMPP-OH can be reacted with diethylenetriamine (DETA) or a similar amine to form a crosslinked epoxy polymer. As shown in FIG. 18: R can represent a hydrogen or methoxy group such that in an epoxy polymer synthesized from DGEDHE-OH:R═H, and in an epoxy polymer synthesized from DGEDMPP-OH:R═OCH₃.

### Example 15

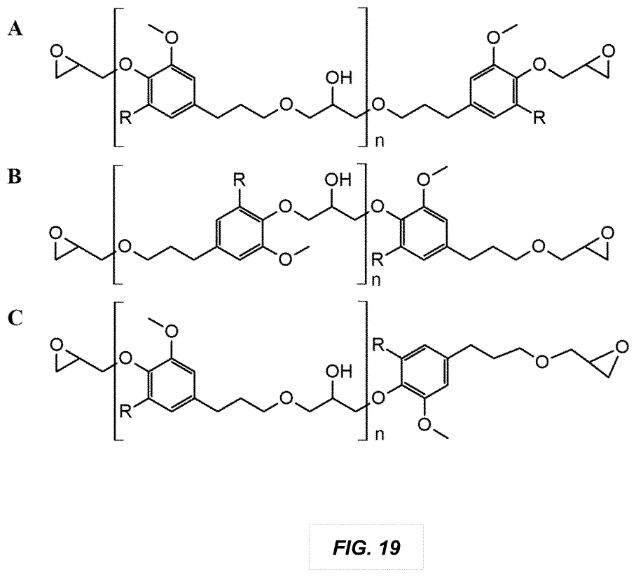

Synthesis of additional epoxy polymers. In this prophetic example, DMPP-OH and DHE-OH monomers are reacted with an excess of an excess of epihydrochlorin (ECH) to produce GEDHE-OH and GEDMPP-OH epoxy resins with terminal glycidylated groups, as shown in FIG. 19. As shown in FIG. 19, R can represent a hydrogen or methoxy group such that in epoxy resins synthesized from DMPP-OH:R═OCH₃, and in epoxy resins synthesized from DHE-OH:R═H.

Having described various systems and methods herein, some embodiments can include, but are not limited to:

In a first embodiment, a catalytic process can include directly converting lignin or lignin-like molecules in a solvent mixture containing aliphatic alcohols into varying ratios of the molecules from the group consisting of 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol) without the addition of any molecular hydrogen.

In a second embodiment, a two-step process can be used whereby a solvent mixture containing aliphatic alcohols is first used to extract lignin from biomass, and the solvent containing the extracted lignin is reacted with a catalyst to produce a mixture of products of varying ratios from the group consisting of 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol).

A third embodiment can include the process of the second embodiment, where the reaction is performed in a single reaction vessel for delignification and hydrodeoxygenation of lignin from a biomass feedstock, the process can include: contacting the biomass or biomass extract with a hydrogenation catalyst and co-catalyst at predetermined process conditions which are modified to produce varying ratios of the molecules from the group consisting of 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol).

A fourth embodiment can include the process of the second embodiment, where the contacting is conducted in a stainless steel pressure reactor and the catalyst in an optional microporous cage or package.

A fifth embodiment can include the process of the second embodiment, where the first step is performed in a steel reactor vessel in which biomass is contained and the second step is contacting is conducted in one of a continuous fixed bed flow reactor or a batch reactor.

A sixth embodiment can include the process of the first or second embodiment, where the contacting is conducted in a mixture of hydrogen, inert gas, or mixed hydrogen and inert gas at a pressure ranging from about 10-90 bar and temperature of about 100° C.-250° C.

A seventh embodiment can include the process of the first or second embodiment, where conditions are optimized to selectively produce DHE and DMPP, the biomass is mixed with 30% or less (by mass) of a catalyst and 40% or less (by mass) of a co-catalyst, where 5-10% catalyst and 10-20% co-catalyst are preferred, and where the biomass can be mixed with 99% or less (by mass) of a catalyst and co-catalyst.

An eighth embodiment can include the process of the first or second embodiment, whereby the solvent is a mixture of ethanol and water which is used as a liquid to extract lignin in a reactor operated at a temperature of 100-250° C. and pressure of 10-50 bar. The temperature and pressure is optimally less than the temperature of the catalytic reactor. The products of the extraction process are then introduced into a second reactor containing a catalyst comprised of nickel and carbon at a preferred temperature of 190-220° C. The products of the second reactor are a mixture from the group consisting of 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol) but predominately DMPP, DHE, DMPP-OH and DHE-OH.

A ninth embodiment can include the process of the eighth embodiment, where the reactors for lignin extraction and catalytic reaction are a combination of: packed bed reactors, constant stirred reactors, any flow reactor, screw extruders, batch, or semi-batch reactors. The preferred process uses a continuous reactor for the lignin extraction from biomass while the hydrodeoxygenation catalyst is contained in a separate packed bed reactor. Multiple reactors in parallel or series can be used for any given process.

A tenth embodiment can include the process of the ninth embodiment, where the effluent of the reactor containing the hydrodeoxygenation catalyst is optionally recycled to the inlet of the reactor to increase the yield and selectivity.

An eleventh embodiment can include the process of the first or second embodiment, where conditions are optimized to selectively produce DHE-OH and DMPP-OH. Wherein the solvent contains 0-100% aliphatic alcohols and 0-100% water. Where 0-30% water, with 70%-100% alcohol is preferred.

A twelfth embodiment can include the process of the first or second embodiment, where conditions are optimized to produce a mixture of DHE, DMPP, DHE-OH, and DMPP-OH. Wherein the biomass is mixed with 1-30% (by mass) hydrogenation catalyst. Where 5-10% catalyst is preferred.

A thirteenth embodiment can include the process of the first or second embodiment, where the catalyst includes a transition metal of Pt, Pd, Ni, Ru, Rh, Ir, Co, Fe, alloys thereof, mixtures thereof.

A fourteenth embodiment can include the process of the first or second embodiment, where the catalyst is a high surface areas nickel sponge catalyst (Raney nickel).

A fifteenth embodiment can include the process of the first or second embodiment, where the co-catalyst is activated carbon, carbon powder, zeolite, or solid acid catalyst.

A sixteenth embodiment can include the process of the first or second embodiment, where the biomass feedstock comprises hardwood, softwood, grasses, maize, and mixtures thereof containing lignin constituent.

A seventeenth embodiment can include the process of the first or second embodiment, where the lignin feedstock is derived from by-products of paper production and biorefineries

An eighteenth embodiment can include the process of the first or second embodiment, where the lignin feedstock contains Kraft lignin, Organosolv lignin, and mixtures thereof.

Various aspects have been disclosed herein. The aspects of the systems and processes can include, but are not limited to:

In a first aspect, a process comprises: contacting a mixture comprising lignin and/or lignin-like molecules with a catalyst to form a reaction mixture, wherein the reaction mixture comprises one or more aliphatic alcohols; and producing one or more reaction products selected from the group consisting of: 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol), and mixtures thereof.

A second aspect can include the process of the first aspect, wherein the contacting occurs without the addition of any molecular hydrogen.

A third aspect can include the process of the first or second aspect, further comprising: extracting the lignin and/or lignin-like molecules from biomass with a solvent mixture comprising the one or more aliphatic alcohols to form a biomass extract, wherein the biomass extract is the mixture comprising the lignin and/or lignin-like molecules.

A fourth aspect can include the process of the third aspect, further comprising: performing the contacting, producing, and extracting are in a single reaction vessel for delignification and hydrodeoxygenation of the lignin and/or lignin like molecules from the biomass.

A fifth aspect can include the process of the fourth aspect, wherein the catalyst comprises a hydrogenation catalyst and a co-catalyst, and wherein the performing comprises: contacting the biomass or the biomass extract with the hydrogenation catalyst and the co-catalyst at predetermined process conditions which are modified to produce varying ratios of the one or more reaction products.

A sixth aspect can include the process of any one of the first to fifth aspects, wherein the contacting is conducted in a stainless steel pressure reactor and the catalyst in an optional microporous cage or package.

A seventh aspect can include the process of the third aspect, wherein the extracting is performed in a steel reactor vessel in which biomass is contained, and wherein the contacting step is conducted in at least one of a continuous reactor or a batch reactor.

An eighth aspect can include the process of any one of the first to seventh aspects, wherein the contacting is conducted in hydrogen, an inert gas, or a mixture of hydrogen and inert gas at a pressure ranging from about 10-90 bar and temperature of about 100° C.-250° C.

A ninth aspect can include the process of any one of the first to eighth aspects, wherein the contacting is conducted at conditions that are optimized to selectively produce DHE and DMPP.

A tenth aspect can include the process of any one of the third to ninth aspects, wherein the biomass is mixed with about 1-30% (by mass) of the catalyst and about 1-40% (by mass) of a co-catalyst.

An eleventh aspect can include the process of any one of the third to ninth aspects, wherein the biomass is mixed with about 5-10% of the catalyst and about 10-20% of a co-catalyst.

A twelfth aspect can include the process of any one of the third to eleventh aspects, wherein the solvent mixture is a mixture of ethanol and water in a liquid phase.

A thirteenth aspect can include the process of the twelfth aspect, wherein the extracting is performed in a reactor operated at a temperature of between about 100-250° C. and pressure of between about 10-50 bar.

A fourteenth aspect can include the process of the twelfth or thirteenth aspect, wherein the extracting is performed in a first reactor and the contacting is performed in a second reactor, and wherein a temperature and pressure of the first reactor is less than a temperature of the second reactor.

A fifteenth aspect can include the process of the fourteenth aspect, wherein the catalyst comprises nickel and carbon.

A sixteenth aspect can include the process of any one of the twelfth to fifteenth aspects, wherein the temperature of the second reactor is between about 190-220° C.

A seventeenth aspect can include the process of any one of the fourteenth to sixteenth aspects, wherein the one or more reaction products comprise a mixture selected from the group consisting of 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol) but predominately DMPP, DHE, DMPP-OH, and DHE-OH.

An eighteenth aspect can include the process of any one of the twelfth to seventeenth aspects, wherein the first reactor and the second reactors are a combination of: packed bed reactors, constant stirred reactors, any flow reactor, screw extruders, batch, or semi-batch reactors.

A nineteenth aspect can include the process of any one of the twelfth to eighteenth aspects, wherein the first reactor comprises a continuous reactor for the extracting, and wherein the second reactor comprises a packed bed reactor comprising the catalyst.

A twentieth aspect can include the process of the eighteenth or nineteenth aspect, wherein the first reactor or the second reactor comprises multiple reactors arranged in parallel or series.

A twenty first aspect can include the process of any one of the twelfth to twentieth aspects, wherein an effluent of the second reactor is recycled to an inlet of the second reactor to increase the yield and selectivity.

A twenty second aspect can include the process of any one of the first to twenty first aspects, wherein reaction conditions are optimized to selectively produce DHE-OH and DMPP-OH.

A twenty third aspect can include the process of the twenty second aspect, wherein the solvent mixture comprises about 0-100% aliphatic alcohols and 0-100% water.

A twenty fourth aspect can include the process of the twenty third aspect, wherein the solvent mixture comprises 0-70% ethanol and/or methanol.

A twenty fifth aspect can include the process of any one of the first to eighth aspects, wherein reaction conditions are optimized to produce a mixture of DHE, DMPP, DHE-OH, and DMPP-OH.

A twenty sixth aspect can include the process of the twenty fifth aspect, wherein the catalyst comprises a hydrogenation catalyst, and wherein the biomass is directly mixed with 1-30% (by mass) of the hydrogenation catalyst.

A twenty seventh aspect can include the process of the twenty fifth aspect, wherein the catalyst comprises a hydrogenation catalyst, and wherein the biomass is directly mixed with 5-10% (by mass) of the hydrogenation catalyst.

A twenty eighth aspect can include the process of any one of the first to twenty seventh aspects, wherein the catalyst comprises a transition metal selected from the group consisting of Pt, Pd, Ni, Ru, Rh, Ir, Co, Fe, alloys thereof, mixtures thereof.

A twenty ninth aspect can include the process of any one of the first to twenty seventh aspects, wherein the catalyst is a high surface areas nickel sponge catalyst (Raney nickel).

A thirtieth aspect can include the process of any one of the first to twenty ninth aspects, wherein the catalyst comprises a co-catalyst, and wherein the co-catalyst comprises activated carbon, carbon powder, zeolite, a solid acid catalyst, or any combination thereof.

A thirty first aspect can include the process of any one of the first to thirtieth aspects, wherein the biomass comprises hardwood, softwood, grasses, maize, or mixtures thereof containing lignin constituent.

A thirty second aspect can include the process of any one of the first to thirty first aspects, wherein the biomass is derived from by-products of paper production and/or biorefineries

A thirty third aspect can include the process of any one of the first to thirty first aspects, wherein the biomass comprises Kraft lignin, Organosolv lignin, or mixtures thereof.

A thirty fourth aspect can include the process of any one of the first to thirty third aspects, further comprising: producing at least one of a phenolic resin or a thermoset from at least a portion of the one or more reaction products.

A thirty fifth aspect can include the process of the thirty fourth aspect, further comprising: mixing the phenolic resin or the thermoset with a cellulosic fiber; and curing the phenolic resin or the thermoset in the cellulosic fiber to form a cross-liked network.

A thirty sixth aspect can include the process of any one of the first to thirty third aspects, further comprising forming at least one of a flavor or fragrance molecule from at least a portion of the one or more reaction products.

In a thirty seventh aspect, a lignin reaction system comprises: biomass, wherein the biomass comprises lignin compounds; a solvent, wherein the solvent comprises one or more aliphatic alcohols; a solid catalyst; one or more reactor vessels, wherein the biomass, the solvent, and the solid catalyst are disposed within the one or more reactor vessels; and one or more reaction products, wherein the one or more reaction products comprise at least one of: 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol), and mixtures thereof.

A thirty eighth aspect can include the system of the thirty seventh aspect, wherein the one or more reaction vessels comprises a single reaction vessel, and wherein the biomass, the solvent, the solid catalyst, and the one or more reaction products are all within the single reaction vessel.

A thirty ninth aspect can include the system of the thirty seventh or thirty eighth aspect, wherein the solid catalyst comprises a hydrogenation catalyst and a co-catalyst.

A fortieth aspect can include the system of any one of the thirty seventh to thirty ninth aspects, wherein the one or more reactor vessels comprise a stainless steel pressure reactor and the catalyst in an optional microporous cage or package.

A forty first aspect can include the system of the thirty seventh aspect, wherein the biomass is disposed within a first reactor vessel of the one or more reactor vessels, and wherein the solid catalyst is disposed in a second reactor vessel of the one or more reactor vessels.

A forty second aspect can include the system of any one of the thirty seventh to forty first aspects, wherein the biomass is mixed with about 1-30% (by mass) of the solid catalyst and about 1-40% (by mass) of a co-catalyst.

A forty third aspect can include the system of any one of the thirty seventh to forty first aspects, wherein the biomass is mixed with about 5-10% of the solid catalyst and about 10-20% of a co-catalyst.

A forty fourth aspect can include the system of any one of the thirty seventh to forty third aspects, wherein the solvent mixture is a mixture of ethanol and water.

A forty fifth aspect can include the system of any one of the thirty seventh to forty fourth aspects, wherein the solid catalyst comprises nickel and carbon.

A forty sixth aspect can include the system of any one of the thirty seventh to forty fifth aspects, wherein the one or more reaction product comprise a mixture selected from the group consisting of 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol) but predominately DMPP, DHE, DMPP-OH, and DHE-OH.

A forty seventh aspect can include the system of the thirty seventh aspect, wherein the one or more reaction vessels comprise a first reactor and a second reactor, and wherein the first reactor and the second reactor are a combination of: packed bed reactors, constant stirred reactors, any flow reactor, screw extruders, batch, or semi-batch reactors.

A forty eighth aspect can include the system of the forty seventh aspect, wherein the first reactor or the second reactor comprises multiple reactors arranged in parallel or series.

A forty ninth aspect can include the system of any one of the thirty seventh to forty eighth aspects, wherein the solvent comprises about 0-100% aliphatic alcohols and 0-100% water.

A fiftieth aspect can include the system of any one of the thirty seventh to forty ninth aspects, wherein the solvent comprises 0-70% ethanol and/or methanol.

A fifty first aspect can include the system of any one of the thirty seventh to fiftieth aspects, wherein the solid catalyst comprises a hydrogenation catalyst, and wherein the biomass is directly mixed with 1-30% (by mass) of the hydrogenation catalyst.

A fifty second aspect can include the system of any one of the thirty seventh to fiftieth aspects, wherein the solid catalyst comprises a hydrogenation catalyst, and wherein the biomass is directly mixed with 5-10% (by mass) of the hydrogenation catalyst.

A fifty third aspect can include the system of any one of the thirty seventh to fifty second aspects, wherein the solid catalyst comprises a transition metal selected from the group consisting of Pt, Pd, Ni, Ru, Rh, Ir, Co, Fe, alloys thereof, mixtures thereof.

A fifty fourth aspect can include the system of any one of the thirty seventh to fifty third aspects, wherein the solid catalyst is a high surface areas nickel sponge catalyst (Raney nickel).

A fifty fifth aspect can include the system of any one of the thirty seventh to fifty fourth aspects, wherein the catalyst comprises a co-catalyst, and wherein the co-catalyst comprises activated carbon, carbon powder, zeolite, a solid acid catalyst, or any combination thereof.

A fifty sixth aspect can include the system of any one of the thirty seventh to fifty fifth aspects, wherein the biomass comprises hardwood, softwood, grasses, maize, or mixtures thereof containing lignin constituent.

A fifty seventh aspect can include the system of any one of the thirty seventh to fifty fifth aspects, wherein the biomass comprises Kraft lignin, Organosolv lignin, or mixtures thereof.

In a fifty eighth aspect, a process comprises: contacting a mixture comprising lignin and/or lignin-like molecules with a catalyst to form a reaction mixture, wherein the reaction mixture, wherein the contacting occurs without any addition of molecular hydrogen; and producing one or more reaction products selected from the group consisting of: 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol), and mixtures thereof.

In a fifty ninth aspect, a process comprises: contacting a mixture comprising lignin and/or lignin-like molecules with a catalyst to form a reaction mixture, wherein the reaction mixture; producing one or more reaction products selected from the group consisting of: 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol), and mixtures thereof; and producing at least one of a phenolic resin or a thermoset from at least a portion of the one or more reaction products.

While preferred embodiments of the invention have been shown and described, modifications thereof can be made by one skilled in the art without departing from the teachings of this disclosure. The embodiments described herein are exemplary only, and are not intended to be limiting. Many variations and modifications of the invention disclosed herein are possible and are within the scope of the invention.

Numerous other modifications, equivalents, and alternatives, will become apparent to those skilled in the art once the above disclosure is fully appreciated. It is intended that the following claims be interpreted to embrace all such modifications, equivalents, and alternatives where applicable. Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

1.-59. (canceled)

60. A lignin reaction system comprising:
biomass, wherein the biomass comprises lignin compounds;
a solvent, wherein the solvent comprises one or more aliphatic alcohols;
a solid catalyst;
one or more reactor vessels, wherein the biomass, the solvent, and the solid catalyst are disposed within the one or more reactor vessels; and
one or more reaction products, wherein the one or more reaction products comprise at least one of: 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol), and mixtures thereof.

61. The system of claim 60, wherein the biomass comprises hardwood, softwood, grasses, maize, Kraft lignin, Organosolv lignin, or mixtures thereof.

62. The system of claim 60, wherein the solid catalyst comprises a hydrogenation catalyst and a co-catalyst.

63. The system of claim 60, wherein the solid catalyst comprises nickel and carbon, or wherein the solid catalyst is a high surface area nickel sponge catalyst.

64. The system of claim 60, wherein the solid catalyst comprises a co-catalyst, and wherein the co-catalyst comprises activated carbon, carbon powder, zeolite, a solid acid catalyst, or any combination thereof.

65. The system of claim 60, wherein the solid catalyst comprises a transition metal selected from the group consisting of Pt, Pd, Ni, Ru, Rh, Ir, Co, Fe, alloys thereof, or mixtures thereof.

66. The system of claim 60, wherein the solvent is a mixture of ethanol and water, or wherein the solvent comprises about 0-100% aliphatic alcohols and 0-100% water.

67. The system of claim 60, wherein the system is not configured to receive any molecular hydrogen.

68. The system of claim 60, wherein the biomass is disposed within a first reactor vessel of the one or more reactor vessels, and wherein the solid catalyst is disposed in a second reactor vessel of the one or more reactor vessels.

69. The system of claim 60, wherein the one or more reaction vessels comprise a first reactor and a second reactor, and wherein the first reactor and the second reactor are a combination of: packed bed reactors, constant stirred reactors, flow reactors, screw extruders, batch, or semi-batch reactors.

70. The system of claim 69, wherein the first reactor or the second reactor comprises multiple reactors arranged in parallel or series.

71. The system claim 69, wherein the first reactor comprises a continuous reactor configured to extract lignin and/or lignin-like molecules from the biomass with the solvent, wherein the second reactor comprises a packed bed reactor comprising the catalyst, and wherein a recycle line is configured to pass an effluent of the second reactor to an inlet of the second reactor.

72. The system of claim 60, wherein the one or more reaction product comprise a mixture of at least two components selected from the group consisting of 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol) but predominately DMPP, DHE, DMPP-OH, and DHE-OH.

73. A process comprising:
contacting a mixture comprising lignin and/or lignin-like molecules with a catalyst to form a reaction mixture, wherein the contacting occurs without any addition of molecular hydrogen; and
producing one or more reaction products selected from the group consisting of: 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol), and mixtures thereof.

74. The system of claim 73, further comprising:
producing at least one of a phenolic resin or a thermoset from at least a portion of the one or more reaction products.

75. The system of claim 74, further comprising:
mixing the phenolic resin or the thermoset with a cellulosic fiber; and
curing the phenolic resin or the thermoset in the cellulosic fiber to form a cross-liked network.

76. A process comprising:
contacting a mixture comprising lignin and/or lignin-like molecules with a catalyst to form a reaction mixture;
producing one or more reaction products selected from the group consisting of: 2-methoxy-4-propylphenol (DHE), 2,6-dimethoxy-4-propylphenol (DMPP), 4-(3-hydroxypropyl)-2,6-dimethoxyphenol (DMPP-OH), 4-(3-hydroxypropyl)-2-methoxyphenol (DHE-OH), 2,6-dimethoxy-4-(prop-1-en-1-yl)phenol (i-DMPP), 2-methoxy-4-(prop-1-en-1-yl)phenol (isoeugenol), and mixtures thereof; and
producing at least one of a phenolic resin or a thermoset from at least a portion of the one or more reaction products.

77. The process of claim 76, further comprising:
producing a thermoset from at least a portion of the one or more reaction products, wherein producing the thermoset comprises at least one of:
1) reacting 4-(3-hydroxypropyl)-2,6-dimethoxyphenol (DMPP-OH), 4-(3-hydroxypropyl)-2-methoxyphenol (DHE-OH), or a combination thereof with epihydrochlorin (ECH) to form the epoxy monomer 3-(3,5-dimethoxy-4-(oxiran-2-ylmethoxy)phenyl)propan-1-ol (DGEDMPP-OH), 3-(3-methoxy-4-(oxiran-2-ylmethoxy)phenyl)propan-1-ol (DGEDHE-OH), or a combination thereof;
2) reacting 4-(3-hydroxypropyl)-2,6-dimethoxyphenol (DMPP-OH), 4-(3-hydroxypropyl)-2-methoxyphenol (DHE-OH), or a mixture thereof with an excess of epihydrochlorin (ECH) to produce 2-((2,6-dimethoxy-4-(3-(oxiran-2-ylmethoxy)propyl)phenoxy)methyl)oxirane (GEDMPP-OH), 2-((2-methoxy-4-(3-(oxiran-2-ylmethoxy)propyl)phenoxy)methyl)oxirane (GEDHE-OH), epoxy resins with terminal glycidylated groups, or combinations thereof.

78. The process of claim 76, further comprising:
reacting at least a portion of the reaction products to form epoxy precursors, wherein reacting at least the portion of the reaction product comprises at least one of:
1) demethylating 4-(3-hydroxypropyl)-2,6-dimethoxyphenol (DMPP-OH) to form 5-(3-hydroxypropyl)benzene-1,2,3-triol (DMPPO-OH), and
reacting the DMPPO-OH with epihydrochlorin (ECH) to form an epoxy monomer comprising 2,2′,2″-(((5-(3-(oxiran-2-ylmethoxy)propyl)benzene-1,2,3-triyl)tris(oxy))tris(methylene))tris(oxirane) (TGEDMPPO-OH), or
2) ortho-demethylating DHE-OH to form 4-(3-hydroxypropyl)benzene-1,2-diol (DHEO-OH), and
reacting the DHEO-OH with epihydrochlorin (ECH) to form an epoxy monomer comprising 2,2′-(((4-(3-(oxiran-2-ylmethoxy)propyl)-1,2-phenylene)bis(oxy))bis(methylene))bis(oxirane) (TGEDHEO-OH).

79. The process of claim 76, further comprising:
reacting 3-(3-methoxy-4-(oxiran-2-ylmethoxy)phenyl)propan-1-ol (DGEDHE-OH), 3-(3,5-dimethoxy-4-(oxiran-2-ylmethoxy)phenyl)propan-1-ol (DGEDMPP-OH), or a mixture thereof with diethylenetriamine (DETA) or an amine to form a crosslinked epoxy polymer.
